(19) United States
(12) Patent Application Publication
(10) Pub. No.: US 2025/0143616 A1
(43) Pub. Date: May 8, 2025

(54) BIOELECTRODE HAVING IMPROVED MECHANICAL AND CHEMICAL DURABILITY AND METHOD FOR MANUFACTURING SAME

(71) Applicant: Daegu Gyeongbuk Institute of Science and technology, Daegu (KR)

(72) Inventors: Sungwon LEE, Dangjin-si, Chungcheongnam-do (KR); Wooseong JEONG, Daegu (KR); Seon Min LEE, Namyangju-si, Gyeonggi-do (KR); Hyeok Joo CHOI, Daegu (KR)

(73) Assignee: Daegu Gyeongbuk Institute of Science and technology, Daegu (KR)

(21) Appl. No.: 18/726,000

(22) PCT Filed: Dec. 29, 2022
(86) PCT No.: PCT/KR2022/021630

§ 371 Date: Jul. 1, 2024

(30) Foreign Application Priority Data
Dec. 29, 2021 (KR) 10-2021-0191083

(51) Int. Cl. A61B 5/265; A61B 5/263; A61B 5/268

(52) U.S. Cl. CPC A61B 5/265; A61B 5/263; A61B 5/268; A61B 2562/125

(57) ABSTRACT

Provided is a bioelectrode having excellent mechanical and chemical durability as well as excellent air permeability and flexibility, and specifically, the bioelectrode includes: a nanofiber elastic mesh sheet including a polymer nanofiber formed by electrospinning; a first metal nanowire network which is embedded on the nanofiber elastic mesh sheet, but is at least partially exposed to the outside; and an uneven layer resulting from a second metal which is placed on the first metal nanowire network exposed to the outside.

### TECHNICAL FIELD

The present invention relates to a bioelectrode having improved mechanical and chemical durability and a method of manufacturing the same, and more particularly, to a bioelectrode which has air permeability and flexibility and also has excellent mechanical and chemical durability and a method of manufacturing the same.

### BACKGROUND ART

A bioelectrode is a device designed in order to send and receive electrical signals to and from body organs and tissues, and is inserted to a human body and/or attached to an epidermis and used for interacting electrically with tissues and cells.

Specifically, the bioelectrode is used for regulating electrical activity of cells and tissues and studying various diseases through electrical therapy, by bringing the bioelectrode into contact with a specific body part to record electrical signals coming from the body for a long period of time or for a short period of time or transmit electrical stimulation to the body.

The bioelectrode is mainly used after being inserted into heart, muscle, brain tissues, and the like which show the physiological state of the body as electrical signals, or after being attached to the epidermis for biosignal monitoring. The bioelectrode is required to have low impedance allowing mediation of minute electrical signals of living organisms, stable interaction with living tissues, excellent biocompatibility, and durability against various deformations such as tensile, shrinkage, distortion, and bending of an electrode, for sophisticated interactions in biological environments, and development of bioelectrode materials is being actively studied in order to satisfy the requirements.

In order to satisfy the requirements, Korean Patent Registration Publication No. 10-1284373 provides a conductive polydimethylsiloxane composite composition containing a conductive filler having an aspect ratio of 1 or more, which may be used as a skin electrode.

However, when an electrode is formed based on polydimethylsiloxane, it is difficult to form a metal layer due to heterogeneity in material aspects such as differences in lattice constant and coefficient of thermal expansion between polydimethylsiloxane which is a silicon-based organic polymer and a metal layer; since adhesive strength between a metal layer and polydimethylsiloxane is weak, when the metal layer is patterned with micron-scale line widths, they are easily separated from each other due to the deformation of the electrode described above; and also, when the electrode is attached to the skin, excretion of sweat produced by the human body or gas permeability is poor, and thus, there is a limitation in monitoring biosignals by attaching the electrode to the skin for a long time.

Thus, development of a bioelectrode which has excellent electrical properties so that biosignals may be stably monitored by inserting and/or attaching to the human body for a long time, has excellent durability and air permeability against deformation of the bioelectrode, has excellent biocompatibility so that it may be used by inserting it to the human body, and has improved chemical durability is needed.

### RELATED ART DOCUMENT

### Patent Document

Korean Patent Registration Publication No. 10-1284373

### DISCLOSURE

### Technical Problem

An object of the present invention is to provide a bioelectrode having excellent air permeability, flexibility, and electrical properties.

Another object of the present invention is to provide a bioelectrode having significantly improved mechanical and chemical durability.

Still another object of the present invention is to provide a method of manufacturing a bioelectrode which is economical and allows easy manufacturing.

### Technical Solution

In one general aspect, a bioelectrode includes: a nanofiber elastic mesh sheet including a polymer nanofiber formed by electrospinning; a first metal nanowire network which is embedded on the nanofiber elastic mesh sheet, but is at least partially exposed to the outside; and an uneven layer resulting from a second metal which is placed on the first metal nanowire network exposed to the outside.

In the bioelectrode according to an exemplary embodiment of the present invention, a contact point between first metal nanowires in the first metal nanowire network may include a melt junction point.

In the bioelectrode according to an exemplary embodiment of the present invention, the first metal nanowire forming the first metal nanowire network may be a silver (Ag) nanowire.

In the bioelectrode according to an exemplary embodiment of the present invention, the second metal placed on the first metal nanowire network exposed to the outside may be any one or more selected from titanium (Ti), tantalum (Ta), platinum (Pt), and gold (Au).

In the bioelectrode according to an exemplary embodiment of the present invention, a loading amount of the first metal nanowire may be 5 to 100 μg per unit area of 1 cm².

In the bioelectrode according to an exemplary embodiment of the present invention, a thickness of the uneven layer resulting from the second metal which is placed on the first metal nanowire network exposed to the outside may be 5 to 150 nm.

In the bioelectrode according to an exemplary embodiment of the present invention, the polymer included in the polymer nanofiber may be one or more selected from an olefin-based elastomer, a styrene-based elastomer, a thermoplastic polyester-based elastomer, a thermoplastic polyurethane-based elastomer, a thermoplastic acryl-based elastomer, a thermoplastic vinyl-based polymer, a thermoplastic fluorine-based polymer, and a mixture thereof.

In the bioelectrode according to an exemplary embodiment of the present invention, a glass transition temperature of the polymer may be 60° C. or lower.

In the bioelectrode according to an exemplary embodiment of the present invention, a diameter ratio of first metal nanowire:polymer nanofiber may be 1:5 to 100.

In the bioelectrode according to an exemplary embodiment of the present invention, a sheet resistance change rate of the bioelectrode supported under vortex conditions in which a fluid is stirred at a speed of 500 to 2000 rpm may be 10% or less as compared with an initial sheet resistance before being supported.

In the bioelectrode according to an exemplary embodiment of the present invention, the fluid may be a liquid phase including any one or more selected from distilled water, a surfactant, and a physiological saline solution.

In the bioelectrode according to an exemplary embodiment of the present invention, the bioelectrode may be a skin-attachable or bioimplantable bioelectrode.

In another general aspect, an electronic skin includes the bioelectrode described above.

In another general aspect, an electronic device includes the bioelectrode described above.

In still another general aspect, a method of manufacturing a bioelectrode is provided.

The method of manufacturing a bioelectrode according to an exemplary embodiment of the present invention may include: a) manufacturing a nanofiber elastic mesh sheet including a polymer nanofiber on a substrate using electrospinning; b) spraying a first metal nanowire ink including a dispersion medium in a droplet form on the nanofiber elastic mesh sheet manufactured on the substrate using a spray method to coat a first metal nanowire; c) optical sintering the first metal nanowire to form a first metal nanowire network in which a portion of the first metal nanowires is embedded on the nanofiber elastic mesh sheet, but at least a portion is exposed to the outside; and d) precipitating a second metal on the first metal nanowire network exposed to the outside by an electroplating method to form an uneven layer.

In the method of manufacturing a bioelectrode according to an exemplary embodiment of the present invention, in b), the first metal nanowire ink may be sprayed in a droplet form in a state in which the substrate is heated to a temperature of 60 to 150° C.

In the method of manufacturing a bioelectrode according to an exemplary embodiment of the present invention, the optical sintering may be performed by irradiating intense pulsed light (IPL).

In the method of manufacturing a bioelectrode according to an exemplary embodiment of the present invention, the intense pulsed light may be irradiation with a light energy of 0.01 to 10 J/cm² for 0.1 to 10 ms.

In the method of manufacturing a bioelectrode according to an exemplary embodiment of the present invention, a contact area between the first metal nanowires in c) may be melt-joined by the optical sintering to form the first metal nanowire network.

In the method of manufacturing a bioelectrode according to an exemplary embodiment of the present invention, the forming of an uneven layer may include: d-1) supporting a cathode electrode and an anode electrode which are the first metal nanowire networks in a precursor solution including the second metal; and d-2) applying voltage to the supported cathode electrode and anode electrode.

In the method of manufacturing a bioelectrode according to an exemplary embodiment of the present invention, the voltage may be applied in a range of 1 to 10 V for 0.5 to 10 minutes.

### Advantageous Effects

Since the bioelectrode according to the present invention includes a nanofiber elastic mesh sheet having a nanomesh structure, it has excellent air permeability and flexibility, and since it includes a first metal nanowire network which is embedded on the nanofiber elastic mesh sheet, but is at least partially exposed to the outside and an uneven layer resulting from a second metal which is placed on the first metal nanowire network exposed to the outside, it may have significantly improved mechanical and chemical durability.

In addition, in the bioelectrode according to the present invention, since the first metal nanowire network includes a melt junction point between nanowires and an uneven layer including a second metal in a portion exposed to the outside is included, the bioelectrode has a decreased contact resistance and an expanded electrical connection path, and may have excellent electrical properties.

Furthermore, improvement of the electrical properties and durability of the bioelectrode according to the present invention is caused by optical sintering and electroplating, and since the manufacturing process is easy, the present invention has an effect of reducing costs and process time incurred.

### DESCRIPTION OF DRAWINGS

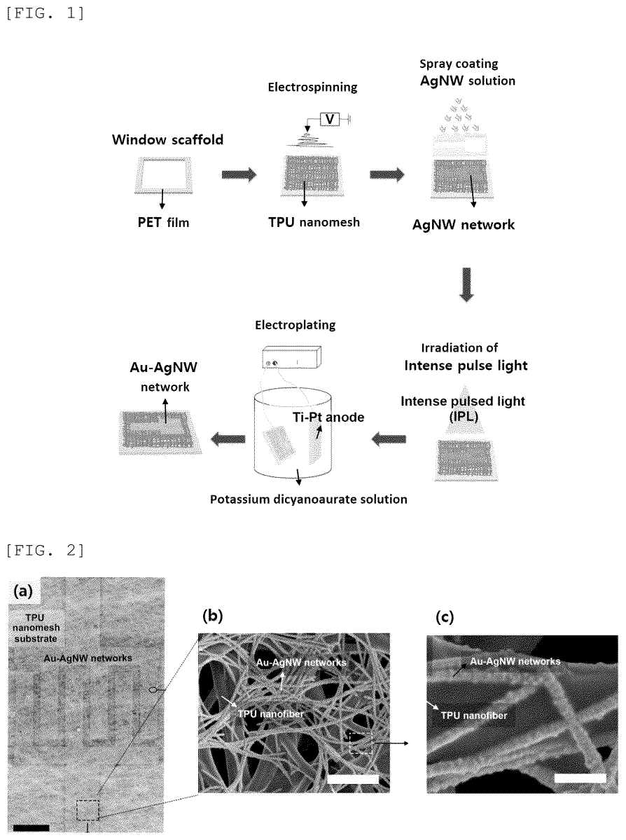

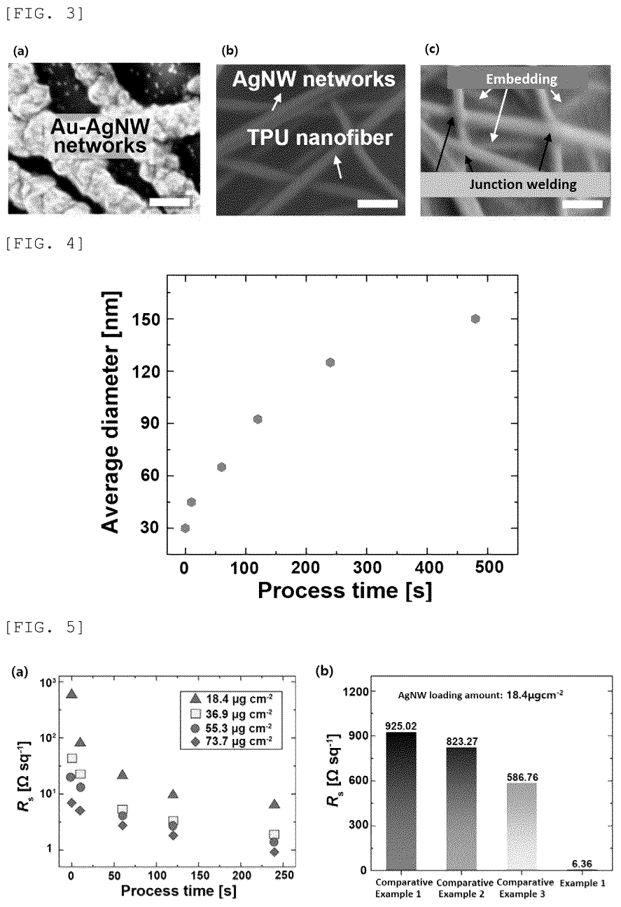

FIG. 1 is a schematic diagram which schematically shows a process of manufacturing a bioelectrode according to an exemplary embodiment of the present invention.
(a), (b), and (c) of FIG. 2 are drawings showing a digital image, a scanning electron microscope (SEM) image in a micrometer scale, and an SEM image in a nanometer scale of Example 1, respectively.
(a), (b), and (c) of FIG. 3 are drawings showing SEM images of Example 1, Comparative Example 1, and Comparative Example 3, respectively.

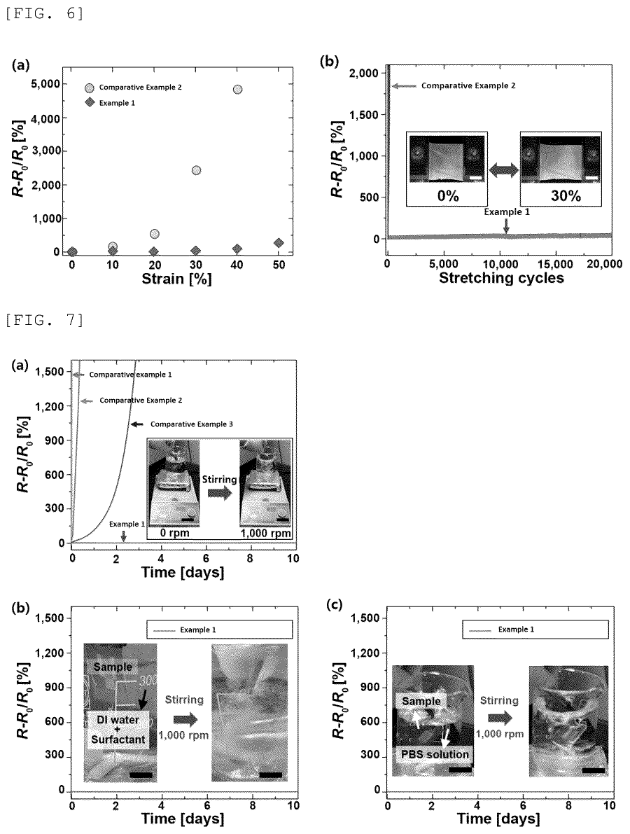

FIG. 4 is a drawing showing results of changes in an average diameter of a nanowire including a gold plating layer over an electroplating process time.
(a) and (b) of FIG. 5 are a drawing showing sheet resistance properties over an electroplating process execution time and a drawing in which sheet resistance properties of Example 1, Comparative Example 1, Comparative Example 2, and Comparative Example 3 including the same loading amount of a silver nanowire are compared, respectively.
(a) and (b) of FIG. 6 are drawings showing sheet resistance changes depending on mechanical deformation degree and results of sheet resistance change trend depending on cyclic strain of tension and contraction for Example 1 and Comparative Example 1, respectively.
(a), (b), and (c) of FIG. 7 are drawings showing results of sheet resistance change trend measured for the bioelectrode of Example 1 and Comparative Examples 1 to 3 under vortex conditions of distilled water, results of sheet resistance change trend of Example 1 under vortex conditions of a mixed solution of distilled water and a surfactant, and results of sheet resistance change trend of Example 1 under vortex conditions of a physiological saline solution, respectively.

### BEST MODE

Hereinafter, the present invention will be described in more detail with reference to specific examples and exemplary embodiments including the accompanying drawings. However, the following specific examples or exemplary embodiments are only a reference for describing the present invention in detail, and the present invention is not limited thereto, and may be implemented in various forms.

In addition, unless otherwise defined, all technical terms and scientific terms have the same meanings as those commonly understood by a person skilled in the art to which the present invention pertains, The terms used for description in the present specification are only for effectively describing a certain specific example, and are not intended to limit the present invention.

In addition, the singular form used in the specification and claims appended thereto may be intended to also include a plural form, unless otherwise indicated in the context.

In addition, unless particularly described to the contrary, “comprising” any elements will be understood to imply further inclusion of other elements rather than the exclusion of any other elements.

A bioelectrode according to an embodiment of the present invention includes: a nanofiber elastic mesh sheet including a polymer nanofiber formed by electrospinning; a first metal nanowire network which is embedded on the nanofiber elastic mesh sheet, but is at least partially exposed to the outside; and an uneven layer resulting from a second metal which is placed on the first metal nanowire network exposed to the outside.

Specifically, since the nanofiber elastic mesh sheet included in the bioelectrode according to an exemplary embodiment of the present invention is formed by electrospinning, it has excellent air permeability and flexibility, and when the bioelectrode is inserted and/or attached to the body, moisture or sweat is easily excreted, harmful gas which may be produced in the human body is easily released, and thus, biosignals may be measured or monitored for a long time, and it is easily fixed to skin or in-vivo tissue and bends or stretches flexibly so that it is not easily detached from the tissue.

In addition, the bioelectrode may have improved physical binding strength to a nanofiber elastic mesh sheet by a first metal nanowire network which is partially embedded on the nanofiber elastic mesh sheet. Furthermore, since the bioelectrode includes an uneven layer resulting from a second metal which is not embedded, that is, exposed to the outside and is placed on the first metal nanowire network, physical binding strength to the nanofiber elastic mesh sheet may be further improved and also chemical durability may be significantly improved by the uneven layer including the second metal.

Therefore, even in the case of applying various deformations such as tension, shrinkage, distortion, and bending to the bioelectrode, the resistance change of the bioelectrode may have reversibility, and when the bioelectrode is restored to its original state after deformation, it is free from a detachment problem between the first metal nanowire network and the uneven layer, may maintain its initial electrical properties, and thus, may have significantly improved mechanical and chemical durability.

As an example, the bioelectrode according to an exemplary embodiment of the present invention may be applied as a skin-attachable or bioimplantable bioelectrode. Specifically, a skin-attachable bioelectrode may be for examining biosignals such as electrocardiogra electromyogram (EMG), or electroencephalogram (EEG), and a bioimplantable bioelectrode may be for stimulating a nervous system tissue or an abnormal tissue such as a tumor. Hereinafter, the bioelectrode according to an embodiment of the present invention will be described in detail for each component.

The bioelectrode according to an exemplary embodiment of the present invention may include a first metal nanowire network which is embedded on the nanofiber elastic mesh sheet, but is at least partially exposed to the outside.

Herein, the first metal nanowire network may have a network structure including a contact point at which first metal nanowires are crossed and in contact with each other.

As an exemplary embodiment, the contact point between the first metal nanowires in the first metal nanowire network may include a melt junction point.

Specifically, since the first metal nanowire network includes a melt junction point, it may have significantly decreased contact resistance to have excellent electrical properties and significantly improve mechanical durability of the bioelectrode.

Conventionally, since conductive fillers having an aspect ratio of 1 or more are electrically connected by a physical contact, the bioelectrode has high contact resistance and has a locally elevated temperature in a contact point due to the high contact resistance, so that is may have reduced durability due to its deterioration. In addition, when the bioelectrode is deformed and then restored, a contact area or a contact degree is changed, so that the resistance of the bioelectrode may be changed as compared with the initial resistance, and as the process is repeated, the electrical properties of the bioelectrode may be eventually very deteriorated.

However, since the bioelectrode according to an exemplary embodiment of the present invention includes a first metal nanowire network in which the contact point between the first metal nanowires includes a melt junction point, contact resistance may be significantly decreased by not a simple physical contact but melt junction, and when the bioelectrode is deformed and then restored, the melt junction point is maintained, so that the initial electrical properties of the bioelectrode are maintained and the mechanical durability of the bioelectrode may be significantly improved as compared with those of the conventional bioelectrode.

In an exemplary embodiment of the present invention, the first metal nanowire network may be partially embedded on the nanofiber elastic mesh sheet.

As a specific example, 20 vol % or more, 30 vol % or more, 40 vol % or more, 50 vol % or more and 90 vol % or less of the first metal nanowire network may be embedded on the nanofiber elastic mesh sheet in the thickness direction.

Since the bioelectrode has a structure in which the first metal nanowire network is partially embedded on the nanofiber elastic mesh sheet in the thickness direction, a strong physical bond between the first metal nanowire and the nanofiber elastic mesh sheet may be formed, whereby the first metal nanowire may have an advantage of not being detached easily from the surface of the nanofiber elastic mesh sheet.

Thus, since the detachment of the first metal nanowire from the nanofiber elastic mesh sheet which may occur due to various deformations such as tension, shrinkage, distortion, and bending of the bioelectrode may be effectively prevented, the mechanical durability of the bioelectrode may be significantly improved.

However, when less than 20 vol % of the first metal nanowire network in the thickness direction of the nanofiber elastic mesh sheet is embedded, a portion of the first metal nanowire network may be released from the nanofiber elastic mesh sheet due to the deformation of the bioelectrode, and when more than 90 vol % of the first metal nanowire network is embedded, thermal deformation may be caused in the nanofiber elastic mesh sheet related to the method of manufacturing a bioelectrode described later, and thus, in order to efficiently improve the mechanical durability of the bioelectrode, it is preferred that the vol % of the first metal nanowire network in the range described above is embedded on the nanofiber elastic mesh sheet in the thickness direction.

As an example, it is advantageous that the first metal nanowire uses a metal having biocompatibility which does not cause rejection or damage to the human body while having excellent conductivity, and as a specific example, the first metal nanowire may be one or more selected from a metal nanowire, a platinum nanowire, and a silver nanowire, and advantageously, may be a silver nanowire considering the electrical properties and economical properties.

In an exemplary embodiment, a loading amount of the first metal nanowire loaded on the nanofiber elastic mesh sheet may be 5 to 100 μg, advantageously 10 to 60 μg, and more advantageously 15 to 50 μg per unit area of 1 cm².

As the loading amount of the first metal nanowire placed on the nanofiber elastic mesh sheet is larger, it may be advantageous in terms of electrical properties, but considering securing the flexibility and air permeability of the bioelectrode and also economic aspect, it is preferred that the loading amount of the first metal nanowire loaded on the nanofiber elastic mesh sheet satisfies the range described above.

As a specific example, the first metal nanowire may have a diameter of 1 to 80 nm, specifically 10 to 60 nm, and more specifically 20 to 50 nm.

As another specific example, the metal nanowire may have an aspect ratio of 100 to 1500, preferably 300 to 1200, and more preferably 500 to 1000.

In order for the melt junction point between the first metal nanowires described above to be sufficiently secured and in order for the first metal nanowire network to be embedded well on the nanofiber elastic mesh sheet, it is preferred that the first metal nanowire has the diameter and the aspect ratio in the above ranges.

In an exemplary embodiment, the first metal nanowire network included in the bioelectrode may be partially embedded on the nanofiber elastic mesh sheet, but is at least partially exposed to the outside, and the bioelectrode may include an uneven layer resulting from a second metal which is placed on the first metal nanowire network exposed to the outside.

By having the structure in which the first metal nanowire network is embedded on the nanofiber elastic mesh sheet, the bioelectrode may have improved mechanical durability, but there is a limit to improving mechanical durability based on physical binding strength to the nanofiber elastic mesh sheet simultaneously with securing flexibility and air permeability of the bioelectrode, chemical durability may be reduced for long term use, and when a precious metal nanowire is used considering only biocompatibility, it is economically disadvantageous.

However, since the bioelectrode according to an exemplary embodiment of the present invention includes the uneven layer resulting from the second metal which is placed on the first metal nanowire network exposed to the outside, the flexibility and air permeability of the bioelectrode may be secured, the physical binding strength to the nanofiber elastic mesh sheet may be further improved, and also, chemical durability may be significantly improved.

Specifically, the nanofiber elastic mesh sheet described later may have a structure in which polymer nanofiber strands are entangled in a network shape, and in the first metal nanowire network which is partially embedded on the nanofiber elastic mesh sheet, but is at least partially exposed, the exposed portion of the first metal nanowire network may be present in various positions such as voids included in the nanofiber elastic mesh sheet and the periphery of the opened polymer nanofiber strands as well as the upper portion of the nanofiber elastic mesh sheet.

Thus, the uneven layer resulting from the second metal which is precipitated and placed, as seen from the method of manufacturing a bioelectrode described later, on the first metal nanowire network exposed to the outside is formed, and physical binding strength to the nanofiber elastic mesh sheet may be further improved by the uneven layer serving as an anchor, and the bioelectrode may have strong binding strength to the first metal nanowire network.

In addition, since the second metal may be a metal having excellent biocompatibility and has strong binding strength to the first metal nanowire network, chemical durability may be significantly improved.

As a specific example, the second metal may be any one or more selected from titanium (Ti), tantalum (Ta), platinum (Pt), and gold (Au), and may be preferably gold (Au) in terms of improving electrical properties.

Herein, the thickness of the uneven layer resulting from the second metal which is placed on the first metal nanowire network exposed to the outside may be 5 to 150 nm, advantageously 10 to 100 nm, and more advantageously 20 to 80 nm.

In order for the uneven layer to serve as an anchor to further improve physical binding strength to the nanofiber elastic mesh sheet to secure the flexibility and air permeability of the bioelectrode and also have excellent mechanical and chemical durability, as described above, it is preferred that the thickness range described above is satisfied.

In an exemplary embodiment, the nanofiber elastic mesh sheet included in the bioelectrode of the present invention may have a structure in which the polymer nanofiber strands formed by electrospinning are entangled in a network shape. Since the bioelectrode includes the nanofiber elastic mesh sheet having a structure in which polymer nanofiber strands are entangled in a network shape, excellent air permeability may be secured, and thus, when the bioelectrode of the present invention is inserted and/or attached to the body, moisture, sweat, and gas produced in the human body may be easily excreted.

In addition, since flexibility may be secured by the structure of the nanofiber elastic mesh sheet in which polymer nanofiber strands are entangled in a network shape, it may be advantageous for fixing the bioelectrode to the skin or in-vivo tissue, and bend or stretch flexibly with movement of the human body so that it is not easily detached from the tissue.

Thus, biosignals may be stably measured and monitored for a long time using the mesh bioelectrode including the nanofiber elastic mesh sheet.

As an exemplary embodiment, the thickness of the nanofiber elastic mesh sheet may be 700 nm to 10 μm, preferably 800 nm to 5 μm, and more preferably 1 to 3 μm. Within the range, excellent flexibility similar to skin may be secured, and attachment to the skin or in-vivo tissue may be easily performed.

In addition, regarding the method of manufacturing a bioelectrode described later, in order to suppress deformation of the nanofiber elastic mesh sheet which may occur by heat, it is preferred that the thickness of the nanofiber elastic mesh sheet satisfies the range.

The nanofiber elastic mesh sheet may include a polymer nanofiber formed by electrospinning, and the formation of the polymer nanofiber by electrospinning will be described in more detail in the method of manufacturing a bioelectrode which is another embodiment of the present invention.

Herein, the polymer included in the polymer nanofiber formed by electrospinning is not particularly limited as long as it may be used in the human body, and as an example, may be one or more selected from olefin-based elastomers, styrene-based elastomers, thermoplastic polyester-based elastomers, thermoplastic polyurethane-based elastomers, thermoplastic acryl-based elastomers, thermoplastic vinyl-based polymers, thermoplastic fluorine-based polymers, and mixtures thereof, but may be selected depending on the use of the bioelectrode, that is, whether the bioelectrode is skin attachable or bioimplantable.

As a specific example, the polymer may be any one or two or more selected from polyvinyl alcohol (PVA), polyethylene glycol (PEG), polypropylene glycol (PPG), polyacrylic acid (PAA), carboxymethyl cellulose (CMC), polyvinylpyrrolidone (PVP), thermoplastic polyurethane (TPU), and polyvinylidene fluoride-trifluoroethylene (PVDF-TrFE).

As an example, the molecular weight of the polymer may be used without a particular limitation as long as the polymer nanofiber may be formed by electrospinning, and specifically, the weight average molecular weight of the polymer may be 10,000 to 500,000 g/mol, preferably 30,000 to 300,000 g/mol, preferably 50,000 to 200,000 g/mol, and most preferably 80,000 to 150,000 g/mol.

In an example of the present invention, the glass transition temperature of the polymer may be 60° C. or lower. Specifically, the glass transition temperature may be 40° C. or lower, more specifically 25° C. or lower, and the lower limit of the glass transition temperature is not particularly limited, but may be −100° C.

When the glass transition temperature of the polymer satisfies the range described above, it may be advantageous for formation of the first metal nanowire network in which a portion of the first metal nanowires is embedded in the nanofiber elastic mesh sheet including the polymer nanofiber in the method of manufacturing a bioelectrode described later.

As an exemplary example, a diameter ratio of first metal nanowire:polymer nanofiber may be 1:5 to 100, preferably 1:10 to 80, and more preferably 1:20 to 60.

As described above, the polymer nanofiber included in the bioelectrode is entangled in a network shape so that the nanofiber elastic mesh sheet may secure air permeability and flexibility, and since the bioelectrode includes the uneven layer placed on the first metal nanowire network in which a portion of the first metal nanowires is embedded on the nanofiber elastic mesh sheet, but at least a portion is exposed, a bioelectrode having excellent mechanical/chemical durability and electrical properties may be provided.

Herein, in order for the bioelectrode including the uneven layer placed on the first metal nanowire network exposed to the outside which is advantageous for embedding of the first metal nanowire on the nanofiber elastic mesh sheet and is formed after the embedding of the first metal nanowire to maintain air permeability and flexibility resulting from the nanofiber elastic mesh sheet, it is preferred that a diameter ratio of first metal nanowire: polymer nanofiber satisfies the range.

As a specific example, the bioelectrode includes voids, and the voids result from the nanofiber mesh sheet on which the first metal nanowire network including the uneven layer is embedded.

As such, since the bioelectrode includes voids, excretion of sweat or gas produced in the human body may be facilitated to allow measurement or monitoring of biosignals for a long time, the voids may be primarily provided from the nanofiber elastic mesh sheet described above in which the polymer nanofiber is entangled in a network form, and the diameter nanowire: polymer nanofiber satisfies the range described above, the voids may be provided even when the uneven layer placed on the first metal nanowire network exposed to the outside which is formed after the first metal nanowire is embedded on the nanofiber elastic mesh sheet is included.

That is, the voids included in the bioelectrode result from the nanofiber mesh sheet on which the first metal nanowire network including the uneven layer is embedded and allows long-term use of the bioelectrode.

As an example, the size of the voids may be 1 nm to 100 μm, specifically 50 nm to 50 μm, and more specifically 100 nm to 10 μm.

As described above, the bioelectrode provided according to an embodiment of the present invention provides excellent air permeability and flexibility and may be used for a long time, has a structure in which the first metal nanowire network in which the contact point between the first metal nanowires includes a melt junction point is partially embedded on the nanofiber elastic mesh sheet, and includes the uneven layer resulting from the second metal which is placed on the first metal nanowire network exposed to the outside, thereby providing significantly improved mechanical and chemical durability as well as excellent sheet resistance properties.

As a specific example, the sheet resistance of the bioelectrode may be 1 to 50Ω/sq, specifically 1 to 30Ω/sq, and more specifically 1 to 10Ω/sq.

Since the contact point between the first metal nanowires is connected not by a simple physical contact but by melt junction, the contact resistance of the first metal nanowire network is significantly decreased, and also, the bioelectrode of the present invention may have low sheet resistance properties in the above range by the uneven layer formed by the second metal which is placed on the first metal nanowire network exposed to the outside.

As an exemplary example, when a strain of 30% is applied to the bioelectrode to perform 20,000 stretching-releasing cycles, a resistance change of the bioelectrode may be 10 times or less, preferably 5 times or less, and more preferably 3 times or less, and the lower limit of the resistance change may be 1.1 times or more, based on the initial resistance value before applying the strain.

Specifically, since the bioelectrode of the present invention includes the structure of the first metal nanowire network including the melt junction point in which the metal nanowire network is partially embedded on the nanofiber elastic mesh sheet, and the uneven layer placed on the first metal nanowire network exposed to the outside, when the bioelectrode is restored to its original state after applying tension, the initial resistance value of the bioelectrode before deformation is similarly maintained.

As such, it is shown that the bioelectrode of the present invention may have excellent mechanical durability so that it may stably perform measurement or monitoring of biosignals even when it is attached to a body part which may be deformed, for example, a joint area.

In an exemplary embodiment, the sheet resistance change rate of the bioelectrode which is supported under the vortex conditions in which a fluid is stirred at a speed of 500 to 2000 rpm may be 10% or less, specifically 5% or less, and more specifically 3% or less, as compared with the initial sheet resistance before being supported.

Herein, the sheet resistance change rate in the range described above may be the sheet resistance change rate of the bioelectrode which is supported for 30 days, substantially 15 days, and more substantially 10 days under the vortex conditions described above.

As a specific example, the stirred fluid may be a liquid phase including any one or more selected from distilled water, a surfactant, and a physiological saline solution.

Herein, the surfactant may be used without a limitation as long as it is a surfactant known in the art having cleaning power, and as a non-limiting example, the surfactant may be an anionic surfactant, a cationic surfactant, a nonionic surfactant, an amphoteric surfactant, and the like, but is not limited thereto.

As described above, it is shown that the bioelectrode according to an exemplary embodiment of the present invention has excellent chemical durability as well as mechanical durability.

According to another embodiment, the present invention provides an electronic skin including the bioelectrode described above.

Though various deformations are applied to the electronic skin including the bioelectrode according to an exemplary embodiment of the present invention, the resistance change of the bioelectrode has reversibility, the electronic skin may effectively detect external stimuli, and it has excellent flexibility and air permeability and may be used for a long time.

In addition, the bioelectrode according to an exemplary embodiment of the present invention has significantly improved chemical durability so that electrical properties are maintained even under the vortex conditions of a solution including a surfactant, and the electronic skin including the bioelectrode may be reused even after washing.

Thus, the electronic skin may be applied to various fields such as internet of things (IoT) which exchanges real-time data over the internet by attaching a sensor to soft robots, prosthetics, health monitoring systems, or objects.

According to another embodiment, the present invention provides an electronic device including the bioelectrode described above.

Specifically, the electronic device including the bioelectrode may be a strain sensor, a pressure sensor, a stretch display, a temperature sensor, a biosignal detection sensor, a circuit interconnection, a gas detection sensor, and the like, but the present invention is not limited thereto.

According to another embodiment, the present invention provides a method of manufacturing a bioelectrode.

The method of manufacturing a bioelectrode according to an exemplary embodiment of the present invention includes: a) manufacturing a nanofiber elastic mesh sheet including a polymer nanofiber on a substrate using electrospinning; b) spraying a first metal nanowire ink including a dispersion medium in a droplet form on the nanofiber elastic mesh sheet manufactured on the substrate using a spray method to coat a first metal nanowire; c) optical sintering the first metal nanowire to form a first metal nanowire network in which a portion of the first metal nanowires is embedded on the nanofiber elastic mesh sheet, but at least a portion is exposed to the outside; and d) precipitating a second metal on the first metal nanowire network exposed to the outside by an electroplating method to form an uneven layer.

As such, the bioelectrode according to an exemplary embodiment of the present invention is manufactured by first manufacturing a nanofiber elastic mesh sheet including a polymer nanofiber by electrospinning, coating the nanofiber elastic mesh sheet with a first metal nanowire using a spray method, performing optical sintering to form a first metal nanowire network in which a portion of the first metal nanowires is embedded on the nanofiber elastic mesh sheet, but at least a portion is exposed to the outside, and precipitating a second metal on the first metal nanowire network which is exposed to the outside by an electroplating method to form an uneven layer, and the manufacture is relatively easy.

In addition, the uneven layer is formed by precipitating the second metal on the first metal nanowire network in which a portion of the first metal nanowires is embedded on the nanofiber elastic mesh sheet by a simple process of optical sintering and electroplating to form the first metal nanowire network, but at least a portion is exposed to the outside, thereby providing a bioelectrode having excellent mechanical and chemical durability as compared with a conventional bioelectrode, with the significantly improved structural stability.

Hereinafter, the method of manufacturing a bioelectrode will be described in more detail for each step, but since the type, the size, and the like of each constituent material are as described above the bioelectrode, overlapping description will be omitted.

First, a step of a) manufacturing a nanofiber elastic mesh sheet including a polymer nanofiber on a substrate using electrospinning is performed.

As an example, the electrospinning may be performed by a method commonly used in the art, and specifically, when the polymer solution is filled into a syringe and high pressure is applied while discharging the solution with a needle tip, the polymer solution which is a liquid phase may be produced into a nanofiber in a collector through electric fields generated at high voltage.

More specifically, the polymer solution according to an exemplary embodiment of the present invention is prepared by dissolving the polymer described above in a solvent, and the polymer may be included at 5 to 30 wt %, preferably 8 to 20 wt %, and most preferably 10 to 15 wt % in the polymer solution. Since the polymer is included in the polymer solution within the range, the nanofiber formed by the electrospinning may form a continuous fiber without breaking into various filaments, which is thus preferred.

Herein, the solvent may be one or more selected from purified water (DI water), acetone, ethanol, N, N-dimethylene acetamide (DMAc), N-methyl-pyrrolidone (NMP), dimethyl sulfoxide (DMSO), methyl ethyl ketone (MEK), and N, N-dimethylformamide (DMF).

In addition, in order to effectively manufacture a nanofiber elastic mesh sheet, a separation distance between a needle tip and a collector, a voltage intensity, and a discharge speed of a polymer solution may be properly adjusted.

As an example, the separation distance between a needle tip and a collector may be 5 to 50 cm, preferably 10 to 40 cm, and more preferably 15 to 30 cm. Herein, when the separation distance between a needle tip and a collector is too close, severe adhesion between nanofibers may occur, and when the separation distance is too far, it may be difficult to form continuous fiber due to evaporation of a solvent, and thus, it is preferred that the separation distance between a needle tip and a collector satisfies the range.

The voltage intensity according to an exemplary embodiment of the present invention is not particularly limited as long as it is a common intensity which is applied for forming a polymer nanofiber through electrospinning, and specifically, for example, the voltage intensity may be 1 to 30 kV, preferably 5 to 25 kV, and more preferably 10 to 20 kV. Within the range, the electrospinning may be effectively performed.

The discharge speed according to an exemplary embodiment of the present invention is for adjusting the thickness of the polymer nanofiber to the target without being broken by adjusting an amount of the discharged polymer solution, and specifically, for example, the discharge speed of the polymer solution may be 0.1 to 20 mL/hour (hr), more preferably 0.5 to 15 mL/hr, and most preferably 1 to 10 mL/hr. Within the range, the polymer nanofiber which is not broken and has a targeted thickness may be easily manufactured.

Subsequently, a step of b) spraying a first metal nanowire ink including a dispersion medium in a droplet form on the nanofiber elastic mesh sheet manufactured on the substrate using a spray method to coat a first metal nanowire is performed.

Specifically, the coating of the first metal nanowire may be performed by exposing only an area to be coated with the first metal nanowire on the nanofiber elastic mesh sheet and coating a partial or entire area of the nanofiber elastic mesh sheet with the first metal nanowire according to the purpose.

As a more specific example, a designed shadow mask is placed on the nanofiber elastic mesh sheet, and an area which is not covered with the shadow mask, that is, is exposed to the outside may be coated with the first metal nanowire. Herein, the first metal nanowire may be one or more selected from a gold nanowire, a platinum nanowire, and a silver nanowire, as described above, and may be preferably a silver nanowire.

As an exemplary embodiment of the present invention, the first metal nanowire may be coated by spraying a first metal nanowire ink including a dispersion medium in a droplet form by a spray method.

Since the first metal nanowire ink is sprayed in a droplet form using a spray method, it may be evenly dispersed on the nanofiber elastic mesh sheet, and agglomeration between the first metal nanowires may be suppressed to facilitate the manufacture of the first metal nanowire network described later.

Herein, the dispersion medium is not particularly limited as long as it is a material known in the art for suppressing agglomeration and also increasing dispersibility of the first metal nanowire, but as an example, may be one or more selected from methanol, ethanol, ethylene glycol, toluene, terpineol, acetonitrile, and isopropanol.

As a specific example, the first metal nanowire ink may include 0.01 to 10 wt %, preferably 0.05 to 5 wt %, and more preferably 0.1 to 1 wt % of the first metal nanowire.

As described above, the diameter of the first metal nanowire may be 1 to 80 nm, specifically 10 to 60 nm, and more specifically 20 to 50 nm, and the aspect ratio of the first metal nanowire may be 100 to 1500, preferably 300 to 1200, and more preferably 500 to 1000.

In order for the first metal nanowire having the morphological characteristics to be evenly dispersed in the first metal nanowire ink including a dispersion medium without agglomeration therebetween and in order for the first metal nanowire to be evenly coated on the nanofiber elastic mesh sheet by spraying the first metal nanowire ink, it is advantageous that the first metal nanowire ink includes the first metal nanowire within the above range.

As an exemplary embodiment, the first metal nanowire ink is sprayed in a droplet form from a spray head to coat the nanofiber elastic mesh sheet with the first metal nanowire.

Herein, the spray head may spray the first metal nanowire ink from a distance of 1 to 50 cm, preferably 10 to 40 cm, and more preferably 20 to 40 cm from the nanofiber elastic mesh sheet, and the spray head may spray the first metal nanowire ink at an angle tilted 0 to 90°, specifically 0 to 80°, and more specifically 0 to 45° to the left or right based on the gravity direction.

In the manufacture of the first metal nanowire network described later by evenly coating the first metal nanowire on the nanofiber elastic mesh sheet, it may be advantageous for forming a melt junction point in a contact point between first metal nanowires and embedding of the first metal nanowire in the thickness direction on the nanofiber elastic mesh sheet, and thus, it is preferred to perform spraying under the conditions described above.

In an exemplary embodiment of the present invention, in the coating of the first metal nanowire in step b), the first metal nanowire ink may be sprayed in a droplet form in the state in which the substrate is heated to a temperature of 60 to 150° C., specifically 70 to 140° C., and more specifically 80 to 130° C.

After manufacturing the nanofiber elastic mesh sheet on the substrate, the first metal nanowire ink is sprayed in a droplet form on the nanofiber elastic mesh sheet in a state in which the substrate is heated to the temperature in the above range, and thus, the dispersion medium included in the droplet is rapidly evaporated to effectively prevent filling of voids included in the nanofiber elastic mesh sheet described above by the droplets and allowing the bioelectrode of the present invention to have excellent air permeability and flexibility. When the dispersion medium is slowly evaporated, agglomeration between the first metal nanowires included in the droplet easily occurs to fill the voids included in the nanofiber elastic mesh sheet, which reduces the air permeability and the flexibility of the bioelectrode, electrical properties may be deteriorated by agglomeration between the first metal nanowires, and thus, it is very important to spray the first metal nanowire ink in a state in which the substrate is heated to the temperature in the above range.

Furthermore, as the substrate is heated, the nanofiber elastic mesh sheet manufactured on the substrate is indirectly heated and deformation by heat is limited, and the first metal nanowire described layer may serve as an advantageous role for being embedded in the thickness direction of the nanofiber elastic mesh sheet.

As an example, the substrate may be any substrate as long as it has a higher glass transition temperature than the polymer nanofiber included in the nanofiber elastic mesh sheet and is a transparent substrate which does not absorb light energy by optical sintering described later, and thus, though the present invention is not limited thereto, the substrate may be selected from polyethylene terephthalate (PET), polyethylene phthalate (PEN), polyetheretherketone (PEEK), polycarbonate (PC), polyarylate (PAR), polyethersulfone (PES), or polyimide (PI).

Subsequently, a step of c) optical sintering the first metal nanowire to form a first metal nanowire network in which a portion of the first metal nanowires is embedded on the nanofiber elastic mesh sheet, but at least a portion is exposed to the outside is performed.

As an exemplary embodiment, the optical sintering may be performed by irradiating intense pulsed light (IPL).

Specifically, the optical sintering instantly raises a surface temperature of the first metal nanowire to 500 to 1500° C. by irradiating light at a desired wavelength in a pulse form for a very short period of time to selectively transfer energy to the first metal nanowire at a rapid speed. A portion of the first metal nanowire may be embedded on the nanofiber elastic mesh sheet by the instantly raised temperature. Herein, the embedding of the first metal nanowire may efficiently occur with the heating effect of the substrate described above as well as the instantly raised surface temperature of the first metal nanowire.

As a specific example, 20 vol % or more, 30 vol % or more, 40 vol % or more, 50 vol % or more and 90 vol % or less of the first metal nanowire network may be embedded in the thickness direction of the nanofiber elastic mesh sheet.

In addition, light welding occurs at a contact point between the first metal nanowires by the raised surface temperature of the first metal nanowire, thereby forming the first metal nanowire network in which the contact point includes a melt junction point.

The bioelectrode of the present invention including the first metal nanowire network including the melt junction point has significantly decreased contact resistance to have excellent sheet resistance properties, and since the vol % of first metal nanowire network in the above range is embedded in the thickness direction of the nanofiber elastic mesh sheet, the bioelectrode of the present invention may maintain the melt junction point as it is even after restoring after deformation of the bioelectrode of the present invention, and since the initial electrical properties of the bioelectrode hardly changes, the mechanical durability of the bioelectrode of the present invention may be improved as compared with the conventional bioelectrode.

As an example, the intense pulsed light may be a light at a wavelength of 300 to 1400 nm, specifically 500 to 1200 nm, and more specifically 800 to 1000 nm.

As a specific example, energy transferred during optical sintering may be adjusted by one or more factors selected from light intensity, light irradiation period, voltage, frequency between pulses, and the number of pulses.

As an example, light irradiated for light energy may be irradiated for 0.1 to 10 ms, preferably 0.5 to 8 ms, and more preferably 1 to 5 ms with a light energy of 0.01 to 10 J/cm², specifically 0.05 to 8 J/cm², and more specifically 0.1 to 4 J/cm².

As an exemplary embodiment, the voltage may be applied at 150 to 500 V, specifically 200 to 400 V, and more specifically 250 to 350V to irradiate light, and herein, the frequency between pulses may be 0.1 to 10 Hz, preferably 0.5 to 5 Hz, and more preferably 0.5 to 1.5 Hz, and the number of pulses may be 10 or less, specifically 5 or less, and more specifically 3 or less.

In order to suppress deformation of the nanofiber elastic mesh sheet by heat applied or accumulated during optical sintering, form the melt junction point by light welding at the contact point between the first metal nanowires described above, and efficiently embed the first metal nanowire network in the thickness direction of the nanofiber elastic mesh sheet, it is preferred to form the first metal nanowire network by performing optical sintering under the conditions.

Subsequently, a step of d) precipitating a second metal on the first metal nanowire network which is not embedded, that is, exposed to the outside by electroplating to form an uneven layer is performed.

In an exemplary embodiment, the forming of an uneven layer may include: d-1) supporting a cathode electrode and an anode electrode which are the first metal nanowire networks in a precursor solution including the second metal; and d-2) applying voltage to the supported cathode electrode and anode electrode.

Herein, the second metal included in the precursor solution may be a metal having excellent biocompatibility.

Specifically, the first metal nanowire network which is partially embedded on the nanofiber elastic mesh sheet is used as a cathode electrode, the cathode electrode is supported in the precursor solution including the second metal, and voltage is applied to precipitate a second metal included in the precursor solution on the first metal nanowire network which is partially embedded on the nanofiber elastic mesh sheet, but is exposed to the outside, thereby forming the uneven layer.

The uneven layer formed on the first metal nanowire network exposed to the outside may serve as an anchor which further improves physical binding strength to the nanofiber elastic mesh sheet, and may have high binding strength to the first metal nanowire network.

Thus, the bioelectrode including the uneven layer including the second metal having excellent biocompatibility which is formed on the first metal nanowire network which is partially embedded on the nanofiber elastic mesh sheet, but is exposed to the outside may have significantly improved chemical durability as well as mechanical durability.

As a specific example, the precursor solution including the second metal may be a metal salt solution including any one or more metals selected from titanium (Ti), tantalum (Ta), platinum (Pt), and gold (Au).

As an advantageous example, the metal salt may be one or more selected from a cyanoaurate salt, and for example, may be one or more selected from potassium dicyanoaurate (I), potassium dicyanoaurate (II), ammonium dicyanoaurate, and sodium dicyanoaurate.

As a specific example, the metal salt solution may include the metal salt at a concentration of 0.01 to 1 g/L, specifically 0.03 to 0.5 g/L, more specifically 0.05 to 0.3 g/L, and still more specifically 0.08 to 0.15 g/L.

In order for the uneven layer formed by precipitation of the second metal included in the precursor solution on the first metal nanowire network exposed to the outside to serve as an anchor which may further improve physical binding strength to the nanofiber elastic mesh sheet, it is advantageous that the concentration of the metal salt solution satisfies the range described above.

As an example, the anode electrode supported in the precursor solution may be used without a limitation as long as it is an insoluble electrode known in the art, and as a non-limiting example, the anode electrode may be selected from Ti/Pt, Ti/IrO₂, Ti/RuO₂, Ti/RuO₂—IrO₂, and the like, but is not limited thereto.

As a specific example, the uneven layer formed by precipitating the second metal on the first metal nanowire network exposed to the outside may be formed by applying voltage of 1 to 10 V, specifically 3 to 8 V for 0.5 to 10 minutes, advantageously 1 to 8 minutes, and more advantageously 2 to 5 minutes to the cathode electrode and the anode electrode supported in the precursor solution.

In order for the bioelectrode including the uneven layer resulting from the second metal precipitated on the first metal nanowire network which is partially embedded on the nanofiber elastic mesh sheet, but is exposed to the outside to have excellent mechanical and chemical durability and also maintain flexibility and air permeability resulting from the nanofiber elastic mesh sheet, it is advantageous that the electroplating process for forming the uneven layer is performed under the conditions described above.

Hereinafter, the bioelectrode and the method of manufacturing the same according to an exemplary embodiment of the present invention will be described in more detail by the following examples. However, the following examples are only a reference for describing the present disclosure in detail, and the present disclosure is not limited thereto and may be implemented in various forms.

In addition, unless otherwise defined, all technical terms and scientific terms have the same meanings as those commonly understood by a person skilled in the art to which the present disclosure pertains. The terms used herein are only for effectively describing a certain exemplary embodiment, and are not intended to limit the present

### DISCLOSURE

### Example 1

Thermoplastic polyurethane (TPU; product No: P22SRNAT, Miractran Co., Ltd.) was mixed with a solvent in which methylethylketone (MKE) and dimethylformamide (DMF) were mixed at a weight ratio of 5:5, and stirring was performed to obtain a TPU polymer solution, the syringe was filled with 11.5 wt % of the TPU polymer solution, and electrospinning was performed on a polyethylene terephthalate (PET) substrate using an electrospinning device (ESR200R2, NanoNC, Korea).

At this time, the inner diameter of the syringe needle was 0.31 mm, a separation distance between the needle tip of the syringe and the PET substrate (collector) was 20 cm, and the TPU polymer solution was spun at a speed of 2 mL/hr for 5 minutes while a voltage of 15 kV was applied, thereby manufacturing a nanofiber elastic mesh sheet in which nanofiber was entangled in a network shape.

Next, a designed shadow mask was placed on the nanofiber elastic mesh sheet, and a silver nanowire ink was sprayed on a partial area of the nanofiber elastic mesh sheet which was not covered by a spray method to coat the nanofiber elastic mesh sheet with silver nanowires. At this time, the silver nanowire ink was prepared by dispersing silver nanowires in ethanol so that 0.5 wt % of silver nanowires are included, and the spraying was performed by setting a spray head to be positioned in parallel to the direction of gravity at a distance separated 30 cm from the center of the nanofiber elastic mesh sheet and applying air pressure of 50 psi to spray the silver nanowire ink at a speed of 0.08 mL/s, thereby coating so that 18.4 μg/cm² of the silver nanowire was loaded. The spraying the silver nanowire ink was performed after placing the substrate on which the nanofiber elastic mesh sheet was manufactured on a hot plate at 110° C.

Thereafter, optical sintering was performed under the conditions of a voltage of 300 V, a light irradiation time of 3 ms, a frequency between pulses of 1 Hz, and the number of pulse of 1 using optical sintering (intense pulsed light sintering) equipment, thereby forming a silver nanowire network so that at least a portion was exposed.

Finally, a gold plating layer was formed on the silver nanowire network exposed using the electroplating method.

The previously formed silver nanowire network as a cathode electrode and Ti—Pt as an anode electrode were supported in a plating bath (0.1 g/L) including potassium dicyanoaurate, a current of 200 mA and a voltage of 5 V were applied for 4 minutes to form a gold plating layer on the exposed silver nanowire network to manufacture a bioelectrode. At this time, the average thickness of the gold plating layer was 62 nm.

FIG. 1 schematically shows a manufacturing process of the bioelectrode according to an exemplary embodiment of the present invention.

### Example 2

The process was performed in the same manner as in Example 1, except that coating was performed so that 36.9 μg/cm² of silver nanowire was loaded on the nanofiber elastic mesh sheet.

### Example 3

The process was performed in the same manner as in Example 1, except that coating was performed so that 55.3 μg/cm² of silver nanowire was loaded on the nanofiber elastic mesh sheet.

### Example 4

The process was performed in the same manner as in Example 1, except that coating was performed so that 73.7 μg/cm² of silver nanowire was loaded on the nanofiber elastic mesh sheet.

### Example 5

The process was performed in the same manner as in Example 1, except that a current of 200 mA and a voltage of 5 V were applied for 8 minutes to form a gold plating layer. At this time, the average thickness of the gold plating layer was 120 nm.

### Comparative Example 1

A bioelectrode was manufactured in the same manner as in Example 1, except that the optical sintering and the electroplating process were excluded.

### Comparative Example 2

A bioelectrode was manufactured in the same manner as in Example 1, except that the optical sintering and the electroplating process were excluded and the heat treatment was performed at a temperature of 150° C. for 30 minutes after coating the silver nanowire on the nanofiber elastic mesh sheet.

### Comparative Example 3

A bioelectrode was manufactured in the same manner as in Example 1, except that the process was performed up to the optical sintering and the electroplating process was excluded.

### Comparative Example 4

A bioelectrode was manufactured in the same manner as in Example 1 except that the optical sintering process was excluded and the electroplating process was performed.

### Comparative Example 5

A bioelectrode was manufactured in the same manner as in Comparative Example 3, except that a nanowire having a core (Ag)-cell (Au) structure was used. At this time, the thickness of the Au shell was 150 nm.

### (Experimental Example 1) Comparison of Morphology Characteristics

The morphology characteristics of the bioelectrodes of Example 1, Comparative Example 1, and Comparative Example 3 were analyzed with a digital camera and a scanning electron microscope (SEM) and the morphology characteristics before and after optical sintering were compared.
(a), (b), and (c) of FIG. 2 are drawings showing a digital image and scanning electron microscope images in a micrometer scale (scale bar=2 μm) and in a nanometer scale (scale bar=300 nm).

Referring to (a), (b), and (c) of FIG. 2, it was confirmed that the bioelectrode of Example 1 was formed in which a portion was embedded on the nanofiber elastic mesh sheet having a structure in which the polymer nanofiber stands were entangled in a network shape and a gold plating layer having an uneven structure was formed on the exposed silver nanowire, and it was found that the bonding part between the nanowires included a melt junction point.
(a), (b), and (c) of FIG. 3 are drawings showing SEM images of Example 1, Comparative Example 1, and Comparative Example 3, respectively.

As shown in (a), (b), and (c) of FIG. 3, unlike Example 1 in which a portion was embedded on the nanofiber elastic mesh sheet, but gold was precipitated on the exposed silver nanowire to form the uneven layer, in Comparative Example 1, the silver nanowire was simply entangled on the nanofiber elastic mesh sheet, not embedded, and in Comparative Example 3, it was confirmed that a portion was embedded on the nanofiber elastic mesh sheet and the melt junction point between silver nanowires was formed.

Since the electroplating process was performed, a portion was embedded on the nanofiber elastic mesh sheet, but gold was precipitated on the exposed silver nanowire and it was confirmed that the gold plating layer had an uneven structure, and thickness of the gold plating layer changed over time of performing the electroplating process. Results of changing the average diameter of the nanowire including the gold plating layer over time of performing electroplating process observed are shown in FIG. 4.

### (Experimental Example 2) Comparison of Electrical Properties

The sheet resistance properties of each of the bioelectrodes manufactured was compared and analyzed.
(a) and (b) of FIG. 5 are a drawing showing sheet resistance properties (including Examples 1 to 4) over an electroplating process execution time and a drawing in which sheet resistance properties of Example 1, Comparative Example 1, Comparative Example 2, and Comparative Example 3 including the same amount of a silver nanowire loaded are compared, respectively.

Referring to (a) of FIG. 5, it was found that as the electroplating process time was increased, the sheet resistance properties were improved, and this is because the average diameter of the nanowire including the gold plating layer was increased to increase electrical connection paths.

In particular, it was observed in Example 1 that the sheet resistance value was about 90 times lower than that before performing the electroplating.

The results may be confirmed by (b) of FIG. 5, and the sheet resistance values of Comparative Examples 1, 2, and 3 in which the same amount of silver nanowires were loaded on the nanofiber elastic mesh sheet were 925.02Ω/sq, 823.27 Ω/sq, and 586.76Ω/sq, respectively, but the sheet resistance value of Example 1 was 6.36Ω/sq, which was found to be significantly lower. It was found that the sheet resistance value of Example 1 was similar to the sheet resistance value when the electroplating process was not performed in Example 4 having the loading amount of the silver nanowires about 4 times larger than Example 1.

This is considered as being due to the fact that the gold plating layer having an uneven structure formed by the electroplating process was formed on the silver nanowire including the melt junction point, thereby increasing the electrical connection path and significantly decreasing contact resistance, so that loss by resistance during charge movement was minimized.

Though not shown in the drawing, it was confirmed that the sheet resistance value of Example 5 in which the electroplating process was performed for 8 minutes was slightly better than that of Example 1.

### (Experimental Example 3) Comparison of Durability Properties

The sheet resistance change rate depending on the mechanical deformation and chemical environments was observed for each of the manufactured bioelectrodes, and the durability characteristics were compared and analyzed.
(a) and (b) of FIG. 6 are drawings showing sheet resistance changes depending on mechanical deformation degree and results of a sheet resistance change trend depending on cyclic strain of tension and contraction for Example 1 and Comparative Example 1, respectively.

At this time, the mechanical deformation degree was measured by fixing both ends of the bioelectrode to a tensile tester, applying stretching deformation, and the cyclic strain (cycle) of tension and contraction was composed of stretching the bioelectrode for 1 second to be 30% deformed, stopping for 1 second, contracting to its original state for the next 1 second, and pausing for the next 1 second and the stretching-releasing cycle was performed 20,000 times.

Referring to (a) of FIG. 6, the sheet resistance change rate of Example 1 was insignificant even when the deformation degree was applied at 50%, but it was found that the sheet resistance value of Comparative Example 2 was increased at a strain of 20% by 500 times or more the initial sheet resistance value before applying the strain.

In addition, as shown in (b) of FIG. 6, it was confirmed that the bioelectrode of Example 1 had almost no sheet resistance change rate even when the tension-releasing cycle to apply a strain of 30% was performed 20, 000 times, but the bioelectrode of Comparative Example 2 had the sheet resistance value which differed by 2,000 times or more compared with the initial sheet resistance value before applying a strain at less than 5 tension-releasing cycles.

It was found therefrom that since the bioelectrode of Example 1 had the gold plating layer having an uneven structure (uneven layer) on the silver nanowire including the melt junction point, it maintained the initial electrical properties before deformation when restored to its original state after performing the tension-releasing cycle, and had significantly better durability against mechanical deformation as compared with the bioelectrode of Comparative Example 2.

Additionally, a chemical durability test was performed for each of the bioelectrodes.

The chemical durability test was performed by supporting each bioelectrode in distilled water, a solution in which a surfactant (commercial laundry detergent; a-sulfo-w-hydroxypoly (oxy-1,2-etyhanediyl) alkyl (c=12, 14) ether, sodium salt (anionic), dodecylbenzenesulfonic acid (anionic), a-dodecyl-w-hydroxy-poly (oxy-1,2-ethanediyl) (nonionic), sodium hydroxide) were mixed with distilled water, and a phosphate buffered saline (PBS), and observing a sheet resistance change trend for 10 days under vortex conditions of the solution which was stirred at a speed of 1,000 rpm using a magnetic bar.
(a), (b), and (c) of FIG. 7 are drawings showing results of sheet resistance change trend measured for the bioelectrode of Example 1 and Comparative Examples 1 to 3 under vortex conditions of distilled water, results of sheet resistance change trend of Example 1 under vortex conditions of a mixed solution of distilled water and a surfactant, and results of sheet resistance change trend of Example 1 under vortex conditions of a physiological saline solution, respectively.

Referring to (a) of FIG. 7, it was confirmed that the bioelectrode of Example 1 had almost no change in the sheet resistance properties for 10 days under the vortex conditions of distilled water, but it was observed that the bioelectrodes of Comparative Examples 1 to 3 had a rapid change in sheet resistance properties on day 1.

In addition, it was found that the bioelectrode of Example 1 had excellent chemical durability properties having almost no change in sheet resistance properties not only under the vortex conditions of distilled water but also under the vortex conditions of the solution including the surfactant and a physiological saline solution, as shown in (b) and (c) of FIG. 7.

Though not shown in the drawing, it was confirmed that the bioelectrodes of Comparative Examples 4 and 5 had a similar sheet resistance change trend similar to Comparative Example 3 under the vortex conditions of distilled water.

It was found therefrom that the bioelectrode of Example 1 had a portion embedded on the nanofiber elastic mesh sheet, but gold was precipitated on the exposed silver nanowire so that the gold plating layer had an uneven structure, and thus, may have excellent mechanical and chemical durability. Specifically, the gold plating layer (uneven layer) formed on the silver nanowire exposed on the nanofiber elastic mesh sheet strengthened binding to the nanofiber elastic mesh sheet in the boundary between an embedded portion and a non-embedded portion of the silver nanowire, so that the bioelectrode of Example 1 had significantly improved mechanical and chemical durability.

Thus, the bioelectrode according to an exemplary embodiment of the present invention had excellent durability against mechanical deformation as well as flexibility and air permeability, may be widely attached to areas from the joint areas causing a high degree of deformation to the skin of the human body and detect and transmit stable biosignals, may be washed and reused due to its excellent chemical durability, and may be inserted into the human body and used.

Hereinabove, although the present invention has been described by the specific matters and specific exemplary embodiments, they have been provided only for assisting in the entire understanding of the present invention. Therefore, the present invention is not limited to the exemplary embodiments, and various modifications and changes may be made by those skilled in the art to which the present invention pertains from this description.

Therefore, the spirit of the present invention should not be limited to the above-described exemplary embodiments, and the following claims as well as all modifications equal or equivalent to the claims are intended to fall within the scope and spirit of the invention.

1. A bioelectrode comprising:
a nanofiber elastic mesh sheet including a polymer nanofiber formed by electrospinning;
a first metal nanowire network which is embedded on the nanofiber elastic mesh sheet, but is at least partially exposed to the outside; and
an uneven layer resulting from a second metal which is placed on the first metal nanowire network exposed to the outside.

2. The bioelectrode of claim 1, wherein a contact point between first metal nanowires in the first metal nanowire network includes a melt junction point.

3. The bioelectrode of claim 1, wherein the first metal nanowire is a silver (Ag) nanowire.

4. The bioelectrode of claim 1, wherein the second metal is any one or more selected from titanium (Ti), tantalum (Ta), platinum (Pt), and gold (Ag).

5. The bioelectrode of claim 1, wherein a loading amount of the first metal nanowire is 5 to 100 μg per unit area of 1 cm².

6. The bioelectrode of claim 1, wherein a thickness of the uneven layer is 5 to 150 nm.

7. The bioelectrode of claim 1, wherein the polymer is one or more selected from an olefin-based elastomer, a styrene-based elastomer, a thermoplastic polyester-based elastomer, a thermoplastic polyurethane-based elastomer, a thermoplastic acryl-based elastomer, a thermoplastic vinyl-based polymer, a thermoplastic fluorine-based polymer, and a mixture thereof.

8. The bioelectrode of claim 7, wherein a glass transition temperature of the polymer is 60° C. or lower.

9. The bioelectrode of claim 1, wherein a diameter ratio of first metal nanowire:polymer nanofiber is 1:5 to 100.

10. The bioelectrode of claim 1, wherein a sheet resistance change rate of the bioelectrode supported under vortex conditions in which a fluid is stirred at a speed of 500 to 2000 rpm is 10% or less as compared with an initial sheet resistance before being supported.

11. The bioelectrode of claim 10, wherein the fluid is a liquid phase including any one or more selected from distilled water, a surfactant, and a physiological saline solution.

12. The bioelectrode of claim 1, wherein the bioelectrode is skin-attachable or bioimplantable.

13. (canceled)

14. An electronic device comprising the bioelectrode of claim 1.

15. A method of manufacturing a bioelectrode, the method comprising:
a) manufacturing a nanofiber elastic mesh sheet including a polymer nanofiber on a substrate using electrospinning;
b) spraying a first metal nanowire ink including a dispersion medium in a droplet form on the nanofiber elastic mesh sheet manufactured on the substrate using a spray method to coat a first metal nanowire;
c) optical sintering the first metal nanowire to form a first metal nanowire network in which a portion of the first metal nanowires is embedded on the nanofiber elastic mesh sheet, but at least a portion is exposed to the outside; and
d) precipitating a second metal on the first metal nanowire network exposed to the outside by an electroplating method to form an uneven layer.

16. The method of manufacturing a bioelectrode of claim 15, wherein in b), the first metal nanowire ink is sprayed in a droplet form in a state in which the substrate is heated to a temperature of 60 to 150° C.

17. The method of manufacturing a bioelectrode of claim 15, wherein the optical sintering is performed by irradiating intense pulsed light (IPL).

18. The method of manufacturing a bioelectrode of claim 17, wherein the intense pulsed light is irradiated with a light energy of 0.01 to 10 J/cm² for 0.1 to 10 ms.

19. The method of manufacturing a bioelectrode of claim 15, wherein a contact area between the first metal nanowires in c) is melt-joined by the optical sintering to form the first metal nanowire network.

20. The method of manufacturing a bioelectrode of claim 15, wherein the forming of an uneven layer includes:
d-1) supporting a cathode electrode and an anode electrode which are the first metal nanowire networks in a precursor solution including the second metal; and
d-2) applying voltage to the supported cathode electrode and anode electrode.

21. The method of manufacturing a bioelectrode of claim 19, wherein the applied voltage is applied in a range of 1 to 10 V for 0.5 to 10 minutes.
